(19) United States
(12) Patent Application Publication
(10) Pub. No.: US 2005/0250147 A1
(43) Pub. Date: Nov. 10, 2005

(54) Digital profiling of polynucleotide populations

(76) Inventor: Stephen C. Macevicz, Cupertino, CA (US)

Correspondence Address: STEPHEN C. MACEVICZ;PARALLELE BIOSCIENCE, INC., 7300 SHORELINE COURT, 2ND FLOOR, SOUTH SAN FRANCISCO, CA, 94080, US

(21) Appl. No.: 11/125,043

(22) Filed: May 9, 2005

Related U.S. Application Data
(60) Provisional application No. 60/569,777, filed on May 10, 2004.

(51) Int. Cl. C12Q 1/68; C12N 9/10

(52) U.S. Cl. 435/6

(57) ABSTRACT

The invention provides methods and compositions for hybridization-based assays that employ oligonucleotide tags, wherein probes specific for the same target polynucleotide are labeled with a plurality of different oligonucleotide tags. When probes are used in conjunction with a microarray, or like, readout platform, containing hybridization sites of tag complements, assay of a target polynucleotide results in a signal being generated from any of a plurality hybridization sites with predetermined addresses and the number of such sites generating a signal is proportional to the relative amount of the target polynucleotide in a population, test sample, or reaction volume, as the case may be. The invention provides methods and compositions for measuring amounts of selected target polynucleotides in a sample and for providing a digital readout of such amounts. Statistical confidence in measurements made by the present invention may be increased as much as desired by increasing the size of the sample of successfully hybridized and selected probes from which signals are generated.

This application claims priority from U.S. provisional patent application Ser. No. 60/569,777 filed 10 May 2004, which application is incorporated herein by reference in its entirety.

### FIELD OF THE INVENTION

The invention relates generally to compositions and methods for analyzing populations of polynucleotides, and more particularly, to the use of tag-containing probes to provide a digital readout of polynucleotide frequency in a population.

### BACKGROUND

The availability of convenient and efficient methods for the accurate identification of genetic variation and expression patterns among large sets of genes is crucial for understanding the relationship between an organism's genetic make-up and the state of its health or disease, Collins et al, Science, 282: 682-689 (1998). In this regard, several powerful techniques have been developed for the analysis of large populations of polynucleotides based either on specific hybridization of probes to microarrays, e.g. Duggan et al, Nature Genetics, 21: 10-14 (1999); Hacia et al, Nature Genetics, 21: 4247 (1999), or on the counting of tags or signatures of DNA fragments, e.g. Velculescu et al, Science, 270: 484487 (1995); Brenner et al, Nature Biotechnology, 18: 630-634 (2000). These techniques have been used in discovery research to identify subsets of genes that have coordinated patterns of expression under a variety of circumstances or that are correlated with, and predictive of events, of interest, such as toxicity, drug responsiveness, risk of relapse, and the like, e.g. Golub et al, Science, 286: 531-537 (1999); Alizadeh et al, Nature, 403: 503-511 (2000); Perou et al, Nature, 406: 747-752 (2000); Shipp et al, Nature Medicine, 8: 68-74 (2002); Hakak et al, Proc. Natl. Acad. Sci., 98: 47454751 (2001); Thomas et al, Mol. Pharmacol., 60: 1189-1194 (2001); De Primo et al, BMC Cancer 2003, 3:3; and the like. Not infrequently the subset of genes found to be relevant has a size in the range of from ten or under to a few hundred.

In addition to gene expression, techniques have also been developed to measure genome-wide variation in gene copy number. For example, in the field of oncology, there is interest in measuring genome-wide copy number variation of local regions that characterize many cancers and that may have diagnostic or prognostic implications, e.g. Albertson et al, Nature Genetics, 34: 369-376 (2003). Presently, genome-wide scans of such variation are carried out using microarrays of BACs containing genomic DNA inserts, e.g. Snijders et al, Nature Genetics, 29: 263-264 (2001); Pinkel et al, Nature Genetics, 20: 207-211 (1998). These microarrays suffer from the same problems of conventional microarrays used for gene expression analysis; thus, measurement of subtle variations in copy number is challenging.

While such hybridization-based techniques offer the advantages of scale and the capability of detecting a wide range of gene expression levels, such measurements are subject to variability relating to probe hybridization differences and cross-reactivity, element-to-element differences within microarrays, and microarray-to-microarray differences, Audic and Clayerie, Genomic Res., 7: 986-995 (1997); Wittes et al, J. Natl. Cancer Inst. 91: 400-401 (1999); Brooks et al, American Pharmaceutical Review, 6: 102-105 (2003).

On the other hand, techniques that provide digital representations of abundance, such as SAGE (Velculescu et al, cited above) or MPSS (Brenner et al, cited above), are statistically more robust; they do not require repetition or standardization of counting experiments as counting statistics are well-modeled by the Poisson distribution, and the precision and accuracy of relative abundance measurements may be increased by increasing the size of the sample of tags or signatures counted, e.g. Audic and Clayerie (cited above).

Both digital and non-digital hybridization-based assays have been implemented using oligonucleotide tags that are hybridized to their complements, typically as part of a detection or signal generation schemes that may include solid phase supports, such as microarrays, microbeads, or the like, e.g. Brenner et al, Proc. Natl. Acad. Sci., 97: 1665-1670 (2000); Church et al, Science, 240: 185-188 (1988); Chee, Nucleic Acids Research, 19: 3301-3305 (1991); Shoemaker et al, Nature Genetics, 14: 450456 (1996); Wallace, U.S. Pat. No. 5,981,179; Gerry et al, J. Mol. Biol., 292: 251-262 (1999); Fan et al, Genome Research, 10: 853-860 (2000); Ye et al, Human Mutation, 17: 305-316 (2001); and the like. A common feature among all of these approaches is a one-to-one correspondence between probe sequences and oligonucleotide tag sequences. That is, the oligonucleotide tags have been employed as probe surrogates for their favorable hybridizations properties, particularly under multiplex assay conditions.

It would be desirable if hybridization-based assays using oligonucleotide tags were available that could measure the expression of moderate numbers of genes or genomic copy number variation at moderate numbers of loci and that could provide a digital readout of such measurements.

### SUMMARY OF THE INVENTION

The present invention is directed to hybridization-based assays that employ oligonucleotide tags such that probes specific for the same target polynucleotide are labeled with a plurality of different oligonucleotide tags. In one particular aspect, each probe in a set of probes has the same target specific moiety, but each is separately labeled with a different oligonucleotide tag. When such an embodiment is used in conjunction with a microarray, or like, readout platform, the detection or measurement of a target polynucleotide results in a signal being generated from any of one or more hybridization sites with predetermined addresses on such an array, and the number of such sites generating a signal is proportional to the relative amount of the target polynucleotide in a population, test sample, or reaction volume, as the case may be.

In one aspect the invention provides a method and composition for determining relative amounts of each of a plurality of polynucleotides in a population. In one embodiment, such a method of the invention comprises the following steps: (i) providing for each target polynucleotide a plurality of probes, each probe of the same plurality being specific for the same target polynucleotide and each probe of the same or different plurality having a different oligonucleotide tag, the oligonucleotide tags of all the pluralities belonging to the same minimally cross-hybridizing set; (ii) combining in a reaction mixture the pluralities of probes with the population so that substantially every target polynucleotide specifically hybridizes to one or more probes of its corresponding plurality and so that probes specifically hybridized to a target polynucleotide are enzymatically modified to form selectable probes; (iii) removing a sample of selectable probes from the reaction mixture; (iv) amplifying and labeling the oligonucleotide tags of the sample; (v) specifically hybridizing the labeled oligonucleotide tags to their respective tag complements on one or more solid phase supports having addressable hybridization sites; and (vi) determining for each plurality of probes a proportion of hybridization sites on the one or more solid phase supports that contain labeled oligonucleotide probes to give a relative amount of the corresponding target polynucleotide in the population.

In another aspect, the invention includes a method of determining absolute concentrations of target polynucleotides in a test sample, wherein in one embodiment such method comprises the following steps: (i) providing one or more nucleic acid standards each having a concentration; (ii) providing for each target polynucleotide and each nucleic acid standard a plurality of probes, each probe of the same plurality being specific for the same target polynucleotide or the same nucleic acid standard and each probe of the same or different plurality having a different oligonucleotide tag, the oligonucleotide tags of all the pluralities belonging to the same minimally cross-hybridizing set; (iii) combining in a reaction mixture the pluralities of probes with the test sample so that substantially every target polynucleotide and nucleic acid standard specifically hybridizes to one or more probes of its corresponding plurality and so that probes specifically hybridized to a target polynucleotide or a nucleic acid standard are enzymatically modified to form selectable probes; (iv) removing a sample of selectable probes from the reaction mixture; (v) amplifying and labeling the oligonucleotide tags of the sample; (vi) specifically hybridizing the labeled oligonucleotide tags to their respective tag complements on one or more solid phase supports having addressable hybridization sites; and (vii) determining an absolute concentration of each target polynucleotide in the test sample by comparing a number of hybridization sites generating signals from a specifically hybridized oligonucleotide tag of a target polynucleotide to a number of hybridization sites generating signals from one or more nucleic acid standards and their respective concentrations.

In another aspect, the invention includes a composition of probes for detecting one or more target polynucleotides in a sample, the composition comprising a plurality of probes for each target polynucleotide, each probe of the same plurality being specific for the same target polynucleotide and each probe of the same or different plurality having a different oligonucleotide tag, wherein each probe specifically hybridizes to a region of a target polynucleotide and the oligonucleotide tags belong to the same minimally cross-hybridizing set.

In one aspect, probes of the invention are molecular inversion probes (described more fully below) and they are converted into selectable probes by a circularization reaction driven by target polynucleotides.

The invention overcomes deficiencies in the prior art by providing compositions and hybridization-based assays for detecting or measuring amounts of selected target polynucleotides in a sample and providing a digital readout of such amounts. Statistical confidence in measurements made by the present invention may be increased as desired simply by increasing the size of the sample of selectable probes from which signals are generated.

### BRIEF DESCRIPTION OF THE FIGURES

FIGS. 1A-1B illustrate the steps of one embodiment of the invention.

FIG. 2 illustrates a method of replicating and labeling an oligonucleotide tag of a selectable probe.

FIG. 3 illustrates a molecular inversion probe that may be used with the invention.

### DEFINITIONS

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Addressable” or “addressed” in reference to tag complements means that the nucleotide sequence, or perhaps other physical or chemical characteristics, of a tag complement can be determined from its address, i.e. a one-to-one correspondence between the sequence or other property of the tag complement and a spatial location on, or characteristic of, the solid phase support to which it is attached. Preferably, an address of a tag complement is a spatial location, e.g. the planar coordinates of a particular region containing copies of the tag complement. However, tag complements may be addressed in other ways too, e.g. by microparticle size, shape, color, signal of micro-transponder, or the like, e.g. Chandler et al, PCT publication WO 97/14028.

“Allele frequency” in reference to a genetic locus, a sequence marker, or the site of a nucleotide means the frequency of occurrence of a sequence or nucleotide at such genetic loci or the frequency of occurrence of such sequence marker, with respect to a population of individuals. In some contexts, an allele frequency may also refer to the frequency of sequences not identical to, or exactly complementary to, a reference sequence.

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRS. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Complementary or substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

“Compexity” or “complex” in reference to mixtures of nucleic acids means the total length of unique sequences in the mixture. In reference to genomic DNA, complexity means the total length of unique sequence DNA in a genome. The complexity of a genome can be equivalent to or less than the length of a single copy of the genome (i.e. the haploid sequence). Estimates of genome complexity can be less than the total length if adjusted for the presence of repeated sequences. In other words, in reference to genomic DNA, “complexity” means the total number of basepairs present in non-repeating sequences, e.g. Wetmur, Critical Reviews in Biochemistry and Molecular Biology, 26: 227-259 (1991); Britten and Davidson, chapter 1 in Hames et al, editors, Nucleic Acid Hybridization: A Practical Approach (IRL Press, Oxford, 1985).

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one aspect, stable duplex means that a duplex structure is not destroyed by a stringent wash, e.g. conditions including temperature of about 5° C. less that the Tₘ of a strand of the duplex and low monovalent salt concentration, e.g. less than 0.2 M, or less than 0.1 M. “Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Fragment”, “segment”, or “DNA segment” refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical or enzymatic in nature. Enzymatic fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3^{rd} Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Where the nucleic acid sample contains RNA, the RNA may be total RNA, poly(A)+ RNA, mRNA, rRNA, or tRNA, and may be isolated according to methods known in the art. See, e.g, Sambrook and Russel., Molecular Cloning: A Laboratory Manual, (Cold Spring Harbor Lab., Cold Spring Harbor, N.Y. 2001). The RNA may be heterogeneous, referring to any mixture of two or more distinct species of RNA. The species may be distinct based on any chemical or biological differences, including differences in base composition, length, or conformation. The RNA may contain full length mRNAs or mRNA fragments (i.e., less than full length) resulting from in vivo, in situ, or in vitro transcriptional events involving corresponding genes, gene fragments, or other DNA templates. In a preferred embodiment, the mRNA population of the present invention may contain single-stranded poly(A)+ RNA, which may be obtained from a RNA mixture (e.g., a whole cell RNA preparation), for example, by affnity chromatography purification through an oligo-dT cellulose column.

“Genetic locus,” or “locus” in reference to a genome or target polynucleotide, means a contiguous subregion or segment of the genome or target polynucleotide. As used herein, genetic locus, or locus, may refer to the position of a nucleotide, a gene, or a portion of a gene in a genome, including mitochondrial DNA, or it may refer to any contiguous portion of genomic sequence whether or not it is within, or associated with, a gene. In one aspect, a genetic locus refers to any portion of genomic sequence, including mitochondrial DNA, from a single nucleotide to a segment of few hundred nucleotides, e.g. 100-300, in length. Usually, a particular genetic locus may be identified by its nucleotide sequence, or the nucleotide sequence, or sequences, of one or both adjacent or flanking regions.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1 M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2^{nd} Ed. Cold Spring Harbor Press (1989) and Anderson “Nucleic Acid Hybridization” 1^{st} Ed., BIOS Scientific Publishers Limited (1999), which are hereby incorporated by reference in its entirety for all purposes above. “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Hybridization-based assay” means any assay that relies on the formation of a stable duplex or triplex between a probe and a target nucleotide sequence for detecting or measuring such a sequence. In one aspect, probes of such assays anneal to (or form duplexes with) regions of target sequences in the range of from 8 to 100 nucleotides; or in other aspects, they anneal to target sequences in the range of from 8 to 40 nucleotides, or more usually, in the range of from 8 to 20 nucleotides. A “probe” in reference to a hybridization-based assay mean a polynucleotide that has a sequence that is capable of forming a stable hybrid (or triplex) with its complement in a target nucleic acid and that is capable of being detected, either directly or indirectly. Hybridization-based assays include, without limitation, assays based on use of oligonucleotides, such as polymerase chain reactions, NASBA reactions, oligonucleotide ligation reactions, single-base extensions of primers, circularizable probe reactions, allele-specific oligonucleotides hybridizations, either in solution phase or bound to solid phase supports, such as microarrays or microbeads. There is extensive guidance in the literature on hybridization-based assays, e.g. Hames et al, editors, Nucleic Acid Hybridization a Practical Approach (IRL Press, Oxford, 1985); Tijssen, Hybridization with Nucleic Acid Probes, Parts I & II (Elsevier Publishing Company, 1993); Hardiman, Microarray Methods and Applications (DNA Press, 2003); Schena, editor, DNA Microarrays a Practical Approach (IRL Press, Oxford, 1999); and the like. In one aspect, hybridization-based assays are solution phase assays; that is, both probes and target sequences hybridize under conditions that are substantially free of surface effects or influences on reaction rate. A solution phase assay may include circumstance where either probes or target sequences are attached to microbeads.

“Kit” refers to any delivery system for delivering materials or reagents for carrying out a method of the invention. In the context of assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials for assays of the invention. In one aspect, kits of the invention comprises one or more pluralities probes each plurality of probes being specific for a different target polynucleotide, such as a genetic locus, a gene expression product, or the like. In another aspect, such probes comprise circularizable padlock probes. Such contents may be delivered to the intended recipient together or separately. For example, a first container may contain an enzyme for use in an assay, while a second container contains probes.

“Ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references, which are incorporated by reference: Whitely et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27: 875-881 (1999); Higgins et al, Methods in Enzymology, 68: 50-71 (1979); Engler et al, The Enzymes, 15: 3-29 (1982); and Namsaraev, U.S. patent publication 2004/0110213.

“Microarray” refers to a solid phase support having a planar surface, which carries an array of nucleic acids, each member of the array comprising identical copies of an oligonucleotide or polynucleotide immobilized to a spatially defined region or site, which does not overlap with those of other members of the array; that is, the regions or sites are spatially discrete. Spatially defined hybridization sites may additionally be “addressable” in that its location and the identity of its immobilized oligonucleotide are known, predetermined, or determinable. Typically, the oligonucleotides or polynucleotides are single stranded and are covalently attached to the solid phase support, usually by a 5′-end or a 3′-end. The density of non-overlapping regions containing nucleic acids in a microarray is typically greater than 100 per cm², and more preferably, greater than 1000 per cm². Microarray technology is reviewed in the following references: Schena, Editor, Microarrays: A Practical Approach (IRL Press, Oxford, 2000); Southern, Current Opin. Chem. Biol., 2: 404-410 (1998); Nature Genetics Supplement, 21: 1-60 (1999). As used herein, “random microarray” refers to a microarray whose spatially discrete regions of oligonucleotides or polynucleotides are not spatially addressed, absent a decoding step to identify the sequence of an immobilized oligonucleotide. That is, the identity of the attached oligonucleotides or polynucleotides is not discernable, at least initially, from its location; it requires a decoding step to determine which probe or tag hybridizes to which site. In one aspect, random microarrays are planar arrays of microbeads wherein each microbead has attached a single kind of hybridization tag complement, such as from a minimally cross-hybridizing set of oligonucleotides. Arrays of microbeads may be formed in a variety of ways, e.g. Brenner et al, Nature Biotechnology, 18: 630-634 (2000); Tulley et al, U.S. Pat. No. 6,133,043; Stuelpnagel et al, U.S. Pat. No. 6,396,995; Chee et al, U.S. Pat. Nos. 6,544,732; 6,620,584; and the like. Likewise, microbeads solid supports, e.g. in a random array, may be identified, or addressable, in a variety of ways, including by optical labels, e.g. fluorescent dye ratios or quantum dots, shape, sequence analysis, radio frequency identification tags, or the like.

“mRNA or mRNA transcripts” include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

“Nucleoside” as used herein includes the natural nucleosides, including 2′-deoxy and 2′-hydroxyl forms, e.g. as described in Komberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6: 855-870 (1996); Mesmaeker et al, Current Opinion in Structual Biology, 5: 343-355 (1995); and the like. Exemplary types of polynucleotides that are capable of enhancing duplex stability include oligonucleotide N3′-P5′ phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999)(two-color real-time PCR). Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Polymorphism” or “genetic variant” means a substitution, inversion, insertion, or deletion of one or more nucleotides at a genetic locus, or a translocation of DNA from one genetic locus to another genetic locus. In one aspect, polymorphism means one of multiple alternative nucleotide sequences that may be present at a genetic locus of an individual and that may comprise a nucleotide substitution, insertion, or deletion with respect to other sequences at the same locus in the same individual, or other individuals within a population. An individual may be homozygous or heterozygous at a genetic locus; that is, an individual may have the same nucleotide sequence in both alleles, or have a different nucleotide sequence in each allele, respectively. In one aspect, insertions or deletions at a genetic locus comprises the addition or the absence of from 1 to 10 nucleotides at such locus, in comparison with the same locus in another individual of a population (or another allele in the same individual). Usually, insertions or deletions are with respect to a major allele at a locus within a population, e.g. an allele present in a population at a frequency of fifty percent or greater.

“Polynucleotide” or “oligonucleotide” are used interchangeably and each mean a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moities, or bases at any or some positions. Polynucleotides typically range in size from a few monomeric units, e.g. 540, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 14 to 36 nucleotides.

“Readout” means a parameter, or parameters, which are measured and/or detected that can be converted to a number or value. In some contexts, readout may refer to an actual numerical representation of such collected or recorded data. For example, a readout of fluorescent intensity signals from a microarray is the address and fluorescence intensity of a signal being generated at each hybridization site of the microarray; thus, such a readout may be registered or stored in various ways, for example, as an image of the microarray, as a table of numbers, or the like. A readout is “digital” when the number or value is obtained by a counting process, e.g. determining a value by counting on a microarray the number of hybridization from which signals are being generated (as distinguished from those sites not generating signals).

“Sample” is used in at least two different contexts in connection with the invention. In one context, “sample,” or equivalently “test sample,” means a quantity of material from a biological, environmental, medical, or patient source in which detection or measurement of target polynucleotides or nucleic acids is sought. It may include a specimen or culture (e.g., microbiological cultures), or other types of biological or environmental samples. A test sample may include a specimen of synthetic origin. Biological test samples may be animal, including human, fluid, solid (e.g., stool) or tissue, as well as liquid and solid food and feed products and ingredients such as dairy items, vegetables, meat and meat by-products, and waste. Biological test samples may include materials taken from a patient including, but not limited to cultures, blood, saliva, cerebral spinal fluid, pleural fluid, milk, lymph, sputum, semen, needle aspirates, and the like. Biological test samples may be obtained from all of the various families of domestic animals, as well as feral or wild animals, including, but not limited to, such animals as ungulates, bear, fish, rodents, etc. Environmental test samples include environmental material such as surface matter, soil, water and industrial samples, as well as test samples obtained from food and dairy processing instruments, apparatus, equipment, utensils, disposable and non-disposable items. These examples are not to be construed as limiting the sample types applicable to the present invention. In the other context, sample refers to a sample of selectable probes isolated from a reaction mixture. That is, it refers to a subset or subpopulation of selectable probes isolated from a reaction mixture that is representative of the full set or population of selectable probes formed in the reaction mixture.

Single-stranded or double-stranded DNA populations according to the present invention may refer to any mixture of two or more distinct species of single-stranded DNA or double-stranded DNA, which may include DNA representing genomic DNA, genes, gene fragments, oligonucleotides, PCR products, expressed sequence tags (ESTs), or nucleotide sequences corresponding to known or suspected single nucleotide polymorphisms (SNPs), having nucleotide sequences that may overlap in part or not at all when compared to one another. The species may be distinct based on any chemical or biological differences, including differences in base composition, order, length, or conformation. The single-stranded DNA population may be isolated or produced according to methods known in the art, and may include single-stranded cDNA produced from a mRNA template, single-stranded DNA isolated from double-stranded DNA, or single-stranded DNA synthesized as an oligonucleotide. The double-stranded DNA population may also be isolated according to methods known in the art, such as PCR, reverse transcription, and the like. Generally, one of ordinary skill in the art will recognize when DNA called for in a process is required to be in single stranded form or double stranded form, such as, when hybridizing a primer to a target polynucleotide or processing a polynucleotide with a restriction endonuclease, respectively. Where the single-stranded DNA population of the present invention is cDNA produced from a mRNA population, it may be produced according to methods known in the art. See, e.g, Maniatis et al. In a preferred embodiment, a sample population of single-stranded poly(A)+ RNA may be used to produce corresponding cDNA in the presence of reverse transcriptase, oligo-dT primer(s) and dNTPs. Reverse transcriptase may be any enzyme that is capable of synthesizing a corresponding cDNA from an RNA template in the presence of the appropriate primers and nucleoside triphosphates. In a preferred embodiment, the reverse transcriptase may be from avian myeloblastosis virus (AMV), Moloney murine leukemia virus (MMuLV) or Rous Sarcoma Virus (RSV), for example, and may be thermal stable enzyme (e.g., hTth DNA polymerase).

“Solid support”, “support”, and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide.

“Specific” or “specificity” in reference to the binding of one molecule to another molecule, such as a labeled target sequence for a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak non-covalent chemical interactions, such as Van der Waal forces, hydrogen bonding, base-stacking interactions, ionic and hydrophobic interactions, and the like, dominate the interaction of the molecules.

“Tₘ” is used in reference to “melting temperature.” Melting temperature is the temperature at which a population of double-stranded nucleic acid molecules becomes half dissociated into single strands. Several equations for calculating the Tm of nucleic acids are well known in the art. As indicated by standard references, a simple estimate of the Tₘ value may be calculated by the equation. Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr., Biochemistry 36, 10581-94 (1997)) include alternative methods of computation which take structural and environmental, as well as sequence characteristics into account for the calculation of Tm.

### DETAILED DESCRIPTION OF THE INVENTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention provides methods and compositions for measuring amounts or concentrations of selected target polynucleotides in a sample. In one aspect, such methods and compositions permit measurement of relative amounts of target polynucleotides within a population and for providing a digital readout of such amounts. In another aspect, such methods and compositions, when used in conjunction with nucleic acid standards of known concentrations, permit measurement of absolute concentrations of target polynucleotides in a test sample.

Target polynucleotides may be the analytes being detected or measured by the method of the invention, such as mRNAs or DNA fragments, or target polynucleotides may be components of other reagents, such as antibody-polynucleotide conjugates, such as disclosed in Hermanson, Bioconjugate Techniques (Academic Press, New York, 1996); Cantor et al, U.S. Pat. No. 5,635,602; and like references. That is, the method of the invention may be employed to measure quantities of non-nucleic acid analytes, such as proteins, in test samples. In one aspect, each binding compound specific for a different antigenic determinant is conjugated to a different polynucleotide. In another aspect, e.g. when polyclonal antibodies are employed, binding compounds specific for the same analyte are each conjugated to the same polynucleotide. Nucleic acid standards are preferably single stranded DNAs have a defined sequence and concentration in a reaction mixture. They may be natural or chemically synthesized, and may be derivatized for convenient manipulation or detection. Preferably, the sequences of nucleic acid standards are sufficiently different from those of target polynucleotides that there is substantially no cross reaction of probes specific for a target polynucleotide and any nucleic acid standard. In one aspect, nucleic acid standards have lengths in the range of from 50 to 1000 nucleotides. Usually, a single nucleic acid standard is employed in an assay of the invention designed to determine concentrations of target polynucleotides.

In still another aspect, the invention provides a digital readout of measurements, such as relative expression levels of selected genes, e.g. based on messenger RNA abundance levels, where such mRNAs are extracted from a biological organism, cell line, cell sample, tissue sample, or the like. In accordance with the invention, target polynucleotides interact with probes of the invention to generate a representative population of selectable probes that each contain an oligonucleotide tag that may be replicated and labeled. The population of selectable probes thus generated is representative in the sense that the amounts of selectable probes specific for given target polynucleotides reflect, i.e. are proportional to, the abundances of those target polynucleotide in the test sample.

An important feature of the invention is the use of probes specific for the same target polynucleotide, but each having a different oligonucleotide tag attached that is subsequently labeled and detected. Thus, in the presence of a single target polynucleotide multiple probes each with a different oligonucleotide tag are converted into selectable probes. In one embodiment, each probe in a plurality of probes of the invention comprises (a) one or more oligonucleotide components that specifically hybridize to the target polynucleotide, and (b) an oligonucleotide tag that is different from all the oligonucleotide tags of probes in the same or different pluralities. The one or more oligonucleotide components of probes within the same plurality may be the same or different. In one embodiment, the oligonucleotide components are identical, and in another embodiment, the oligonucleotide components may be specific for different regions of the same target polynucleotide.

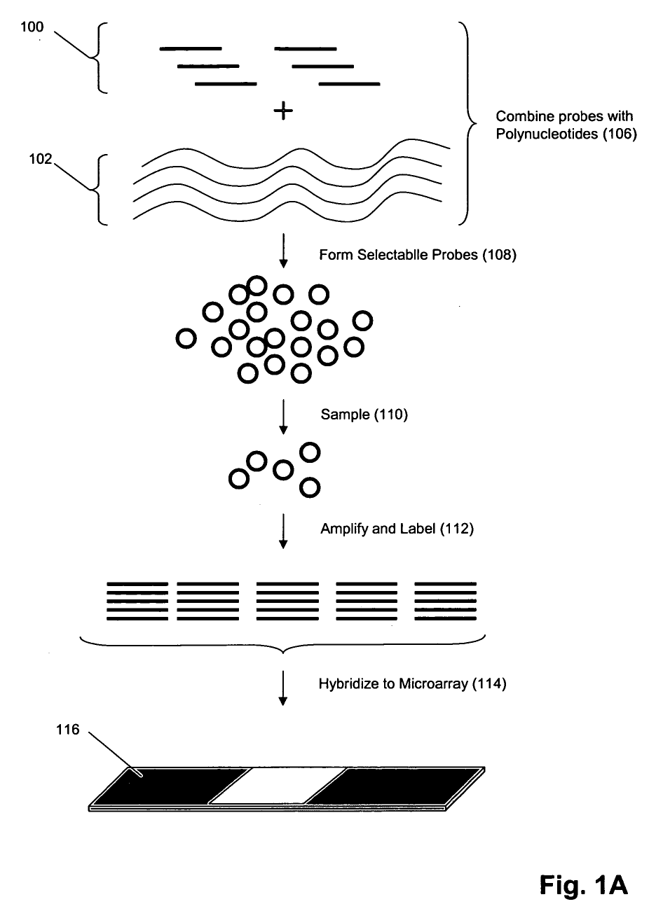

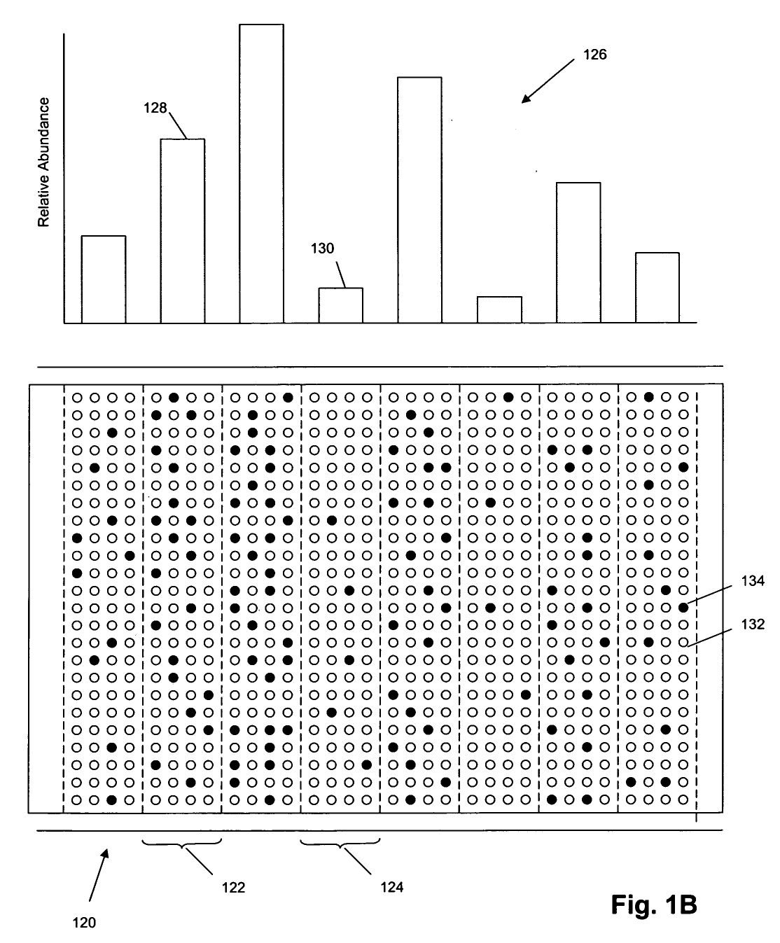

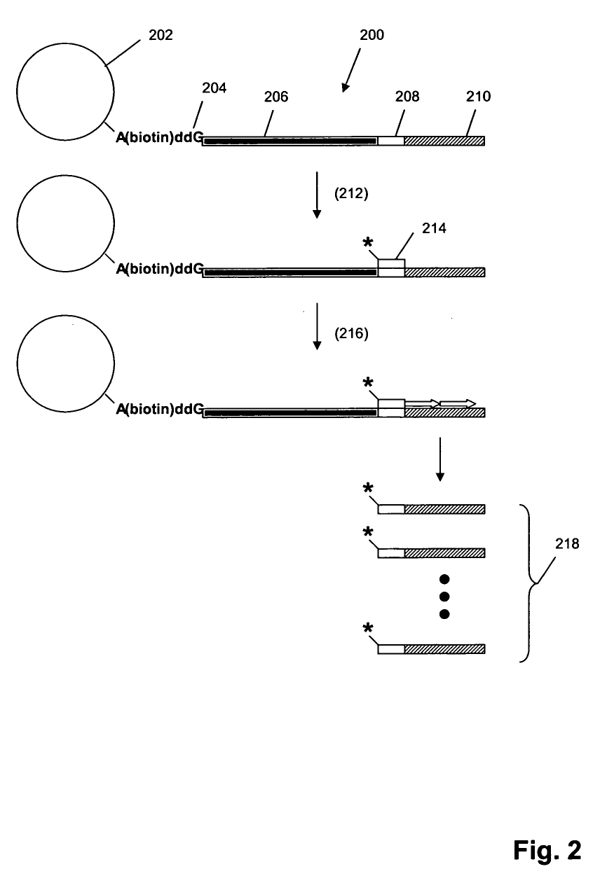

An embodiment of the invention is illustrated in FIGS. 1A-1B. Probes (100), e.g. molecular inversion probes, are combined (106) with target polynucleotides (102), e.g. mRNA extracted from a cell line or tissue using conventional techniques, so that probes (100) can specifically hybridize to their respective targets in the reaction mixture. Depending on the type of probe employed, further reaction steps may be required to form selectable probes (108). For example, in the case of molecular inversion probes, after specific hybridization, probes are extended with a nucleic acid polymerase lacking 5′→3′ exonuclease activity, ligated to form circular DNA molecules, and finally, the reaction mixture is treated with one or more exonucleases and/or Rnases to digest any non-circular polynucleotides. A sample of selectable probes is isolated (110), after which the selectable probes are amplified and labeled (112). A variety of sample techniques may be employed depending on the nature of the selectable probes. For example, if the selectable probe has a capture moiety, e.g. a biotin, then sampling can take place by using solid phase capture, e.g. avidinated magnetic beads. Either a known number of magnetic beads may be combined with the selectable probes, and/or free biotin can be added to the reaction mixture so that selectable probes and free biotin compete for binding sites on the magnetic beads. By adjusting the concentration of biotin appropriately, the magnetic beads will bind a sample of selectable probes of a desired size. FIG. 2 illustrates the capture of selectable probes (200) by magnetic bead (202). In this illustration, selectable probe (200) has a target polynucleotide binding region (206) that is extended when hybridized to its respective target polynucleotide, shown in FIG. 2 as being extended with a biotinylated dideoxyguanosine, a primer binding region (208), and an oligonucleotide tag (210). Primer (214) is hybridized (212) to primer binding region (208) and is extended (216) in a conventional “linear” PCR to generate a collection of label oligonucleotide tags (218), which are hybridized to a microarray of tag complements for a readout.

Alternatively, for example in the case of molecular inversion probes that do not have a capture moiety, a dilution series may be formed from the reaction mixture. For example, a 4 μL sample may be taken from the original reaction mixture, typically 40 μL, and diluted by a factor of ten. This can be repeated for 4-5 times, depending on factors including the initial concentration of probes, the efficiency of converting probes into selectable probes, and the like. A sample, e.g. 1-10 μL, is removed from the final dilution and the oligonucleotide tags in the sample are amplified, e.g. using PCR, as disclosed by Hardenbol et al., Nature Biotechnology, 21: 673-678 (2003). After such amplification, the labeled oligonucleotide tags are hybridized (114) to a microarray (116) of tag complements.

Since the relative abundances of the target-specific sequences of the selectable probes is representative of the relative abundances of their respective target polynucleotides, then the numbers of selectable probes of each type will be proportional to the number of target polynucleotides of that type in the original population of target polynucleotides. Thus, when labeled oligonucleotide tags are specifically hybridized to a microarray of tag complements, a proportion of the hybridization sites for a particular selectable probe type that will contain labeled oligonucleotide tags reflects the relative abundance of the target polynucleotide and provides a convenient digital readout of such abundance, as is illustrated in FIG. 1B. A portion (120) of a microarray of tag complements is shown, wherein the hybridization sites on the microarray containing the tag complements are shown as open circles (132) or filled circles (134), depending on whether no labeled tags or labeled tags are specifically hybridized. For the sake of illustration, all of the hybridization sites associated with a particular selectable probe have been grouped into bands, e.g. (122) and (124). Although such an arrangement could be employed, it is not necessary that the hybridization sites of a particular selectable probe type occupy the same region of a microarray. As illustrated, each band has a number of hybridization sites occupied with labeled probe that is proportional to the abundance of its corresponding target polynucleotide in a test sample, which relative abundances are illustrated in bar graph (126). The relative heights of the bars, e.g. (128) and (130), are proportional to the number of hybridization sites displaying label, e.g. (122) and (124), respectively.

Selectable probes result, or are formed, from probes that have been modified in a reaction as a result of specifically hybridizing to a target polynucleotide. In one aspect, such specific hybridization creates a substrate for an enzyme that modifies the probe, either to implement a first step in conversion to a selectable probe, or to bring about a complete conversion to a selectable probe. Such modification in whole or in part confers a property on the modified probe that allows it to be selected, or removed from, unmodified probes. For example, such selection may be effected by removal or separation from unmodified probes, by destruction of unmodified probes, or by other such means. Modifications may be carried out chemically or enzymatically. Usually, probes are modified enzymatically, such as by ligation, extension with a polymerase, or the like. In one aspect, probes are modified by ligation so that they form closed circular DNAs. In another aspect, probes are extended by a nucleic acid polymerase to incorporate a modified nucleotide that contains a capture moiety, such as biotin. In another aspect, both of the above modifications are accomplished by one or more template-driven enzymatic reactions. Exemplary probes include molecular inversion probes, padlock probes, rolling circle probes, ligation-based probes with “zip-code” tags, single-base extension probes, and the like, e.g. Hardenbol et al, Nature Biotechnology, 21: 673-678 (2003); Nilsson et al, Science, 265: 2085-2088 (1994); Baner et al, Nucleic Acids Research, 26: 5073-5078 (1998); Lizardi et al, Nat. Genet., 19: 225-232 (1998); Gerry et al, J. Mol. Biol., 292: 251-262 (1999); Fan et al, Genome Research, 10: 853-860 (2000); International patent publications WO 2002/57491 and WO 2000/58516; U.S. Pat. Nos. 6,506,594 and 4,883,750; and the like, which references are incorporated herein by reference. In one aspect, probes of the invention are molecular inversion probes, e.g. as disclosed in Hardenbol et al (cited above) and in Willis et al, Internation patent publication WO 2002/057491. In the case of molecular inversion probes, selectable probes are formed by circularizing probes in a template-driven reaction on a target polynucleotide followed by digestion of non-circularized polynucleotides, such as target polynucleotides, unligated probe, probe concatatemers, and the like, with an exonuclease. In another aspect, probes of the invention comprise an oligonucleotide tag and a target-specific region that is extended by a polymerase reaction to add a nucleotide with a capture moiety, such as biotin, as disclosed in Fan et al (cited above) and Mao et al (cited above). Selectable probes are formed by capturing extended probes on a solid phase support derivatized with a capture agent, e.g. avidinated magnetic microbeads, and separating them from the reaction mixture.

Many different terminator-capture moiety combinations are available. Preferably, dideoxynucleoside triphosphates are used as terminators. In one aspect, capture moieties may be attached to such terminators derivatized with an alkynylamino group, as taught by Hobbs et al, U.S. Pat. No. 5,047,519 and Taing et al, International patent publication WO 02/30944, which are incorporated herein by reference. Preferable capture moieties include biotin or biotin derivatives, such as desbiotin, which are captured with streptavidin or avidin or commercially available antibodies, and dinitrophenol, digoxigenin, fluorescein, and rhodamine, all of which are available as NHS-esters that may be reacted with alkynylamino-derivatized terminators. These reagents as well as antibody capture agents for these compounds are available for Molecular Probes, Inc. (Eugene, Oreg.).

Generally, probes of the invention specifically hybridize to their corresponding target polynucleotides in a region having a length in the range of from 9 to 100 nucleotides. Usually, such region is contiguous; however, in some embodiments, a probe may bind to two non-continuous regions, e.g. as with gap-ligation probes, as disclosed in Abravaya et al, Nucleic Acids Research, 23: 675-682 (1995); or in Hardenbol et al (cited above). Probes of the invention comprise oligonucleotides that are made by conventional methodologies, e.g. by direct synthesis, or for longer probes, convergent synthesis, e.g. as disclosed in Namsaraev, U.S. patent publication 2004/0110213, which is incorporated herein by reference.

After selectable probes are formed, a sample of such probes are isolated from the reaction mixture. In one aspect, the size of the sample is large enough so that the total number of selectable probes in the sample is less than the total number of oligonucleotide tags in the tag repertoire being used. In another aspect, the total number of selectable probes in a sample is substantially less than the size of the tag repertoire, for example, eighty percent, or sixty percent, or fifty percent, or forty percent. Usually, the size of the tag repertoire, i.e. the number of oligonucleotide tags having different sequences, is the same as the number of elements in the microarray being employed as a readout device (assuming, of course, that each element, or hybridization site, of the microarray contains a tag complement of only a single oligonucleotide tag, and that tag complements at different sites have different sequences). Generally, a sample is sufficiently large so as to obtain a statistically significant representation of selectable probes specific for the target polynucleotides whose quantities are being measured. On the other hand, the sample should not be so large as to contain substantially every oligonucleotide tag in the repertoire. In the latter case, all of the hybridization sites of a microarray would be occupied; therefore, no useful information would be obtained about relative abundances. For example, the relative abundances of 50 target polynucleotides may be measured in the following system: a tag repertoire of 10,000 oligonucleotide tags and a microarray of 10,000 tag complements are provided; 10,000 probes are synthesized such that each probe has a different oligonucleotide tag and such that there are groups of 200 probes each that are all specific for the same target polynucleotide. After selectable tags are formed in a reaction mixture, a portion of the reaction mixture containing 5000 selectable probes are removed, the oligonucleotide tags of the selectable probes are replicated and label, and the labeled oligonucleotide probes are hybridized to microarray of tag complements. By sampling statistics, about 50 percent of the 5000 selectable probe will have unique oligonucleotide tags, e.g. Brenner et al, U.S. Pat. No. 5,846,719; thus, about 2500 hybridization sites will be occupied after hybridization. If the 50 target polynucleotide have equal abundances, then 50 of the 200 hybridization sites corresponding to each target polynucleotide should be occupied. Likewise, if target polynucleotides 1-25 were each equally expressed at a level one third that of every target polynucleotide 26-50, also equally expressed, then 625 (25×25) hybridization sites of target polynucleotides 1-25 would be occupied and 1875 (75×25) hybridization sites of target polynucleotides 26-50 would be occupied.

In one aspect of the invention, methods are implemented by providing a set, or plurality, of probes for each polynucleotide whose abundance is to be measured. Each probe comprises a polynucleotide having at least one region (“target complementary region” or “target specific region”) that is complementary the target polynucleotide corresponding to the probe's set, or plurality. As used herein, the term “target polynucleotide” refers to one of a plurality of polynucleotides in a population whose abundances, particularly relative abundances, are to be determined. As mentioned above, target specific regions of probes from the same set may all be complementary to the same portion of a target polynucleotide or may be complementary to different portions. Usually, if target specific regions of probes of the same set are complementary to different portions of a target polynucleotides, such portions do not overlap. Each probe further comprises an oligonucleotide tag that preferably is not complementary to any of the polynucleotides in the population. Each probe within all the different sets, or pluralities, has a different oligonucleotide tag. Probes may also contain additional element, e.g. RNA polymerase binding site, for permitting oligonucleotide tags to be replicated and labeled using conventional techniques, such as disclosed in Mao et al, International Patent Publication WO 02/097113.

The number of probes in each set is a plurality that may vary widely depending on several factors including, but not limited to, the precision required in the measurements, whether there is a wide dynamic range of expression levels or abundances that must be measured, the availability and cost of providing large microarrays for a readout, and the like. In one aspect, the number of probes in each set is in a range from 2 to about 10,000; or from 2 to about 1000; or from 2 to 100. In another aspect, the number of probes in each set is in a range from 10 to about 10,000; or from 10 to about 1000; or from 10 to 100. The number of probes within one set may be the same or different than the number of probes in other sets.

### Oligonucleotide Tags and Minimally Cross-Hybridizing Sets

In one aspect, the invention employs minimally cross-hybridizing sets of oligonucleotide tags, such as disclosed in Brenner et al, U.S. Pat. No. 5,846,719; Mao et al (cited above); Fan et al, International patent publication WO 2000/058516; Morris et al, U.S. Pat. No. 6,458,530; Morris et al, U.S. patent publication 2003/0104436; Church et al, European patent publication 0 303 459; Huang et al, U.S. Pat. No. 6,709,816; which references are incorporated herein by reference. The sequences of oligonucleotides of a minimally cross-hybridizing set differ from the sequences of every other member of the same set by at least two nucleotides, and more preferably, by at least three nucleotides. Thus, each member of such a set cannot form a duplex (or triplex) with the complement of any other member with less than two mismatches, or three mismatches as the case may be. Preferably, perfectly matched duplexes of tags and tag complements of the same minimally cross-hybridizing set have approximately the same stability, especially as measured by melting temperature. Complements of oligonucleotide tags, referred to herein as “tag complements,” may comprise natural nucleotides or non-natural nucleotide analogs. In one aspect, non-natural nucleic acid analogs are used as tag complements that remain stable under repeated washings and hybridizations of oligonucleotide tags. In particular, tag complements may comprise peptide nucleic acids (PNAs). Oligonucleotide tags from the same minimally cross-hybridizing set when used with their corresponding tag complements provide a means of enhancing specificity of hybridization. Microarrays of tag complements are available commercially, e.g. GenFlex Tag Array (Affymetrix, Santa Clara, Calif.); and their construction and use are disclosed in Fan et al, International patent publication WO 2000/058516; Morris et al, U.S. Pat. No. 6,458,530; Morris et al, U.S. patent publication 2003/0104436; and Huang et al (cited above).

As mentioned above, in one aspect tag complements comprise PNAs, which may be synthesized using methods disclosed in the art, such as Nielsen and Egholm (eds.), Peptide Nucleic Acids: Protocols and Applications (Horizon Scientific Press, Wymondham, UK, 1999); Matysiak et al, Biotechniques, 31: 896-904 (2001); Awasthi et al, Comb. Chem. High Throughput Screen., 5: 253-259 (2002); Nielsen et al, U.S. Pat. No. 5,773,571; Nielsen et al, U.S. Pat. No. 5,766,855; Nielsen et al, U.S. Pat. No. 5,736,336; Nielsen et al, U.S. Pat. No. 5,714,331; Nielsen et al, U.S. Pat. No. 5,539,082; and the like, which references are incorporated herein by reference. Construction and use of microarrays comprising PNA tag complements are disclosed in Brandt et al, Nucleic Acids Research, 31(19), e119 (2003).

Preferably, oligonucleotide tags and tag complements are selected to have similar duplex or triplex stabilities to one another so that perfectly matched hybrids have similar or substantially identical melting temperatures. This permits mismatched tag complements to be more readily distinguished from perfectly matched tag complements in the hybridization steps, e.g. by washing under stringent conditions. Guidance for carrying out such selections is provided by published techniques for selecting optimal PCR primers and calculating duplex stabilities, e.g. Rychlik et al, Nucleic Acids Research, 17: 8543-8551 (1989) and 18: 6409-6412 (1990); Breslauer et al, Proc. Natl. Acad. Sci., 83: 3746-3750 (1986); Wetmur, Crit. Rev. Biochem. Mol. Biol., 26: 227-259 (1991); and the like. A minimally cross-hybridizing set of oligonucleotides can be screened by additional criteria, such as GC-content, distribution of mismatches, theoretical melting temperature, and the like, to form a subset which is also a minimally cross-hybridizing set.

### Hybridization-Based Assays

As mentioned above, the invention relates to the use of hybridization-based assays to detect or measure interfering polymorphic loci. Such assays are widely used in multiplexed formats to simultaneously genotype DNA samples at multiple loci, e.g. allele-specific muliplex PCR, arrayed primer extension (APEX) technology, variation detection arrays, solution phase primer extension or ligation assays, and the like, described in the following references: Shumaker et al, Hum. Mut., 7: 346-354 (1996); Cronin et al, U.S. Pat. No. 6,468,744; Huang et al, U.S. Pat. Nos. 6,709,816 and 6,287,778; Fan et al, U.S. patent publication 2003/0003490; Chee et al, U.S. Pat. No. 6,355,431; Gunderson et al, U.S. patent publication 2005/0037393; Hacia et al, U.S. Pat. No. 6,342,355; Kennedy et al, Nature Biotechnology, 21: 1233-1237 (2003); Chou et al, Clin. Chem., 49: 542-551 (2003); and the like.

In one aspect, hybridization-based assays include circularizing probes, such as padlock probes, rolling circle probes, molecular inversion probes, linear amplification molecules for multiplexed PCR, and the like, e.g. padlock probes being disclosed in U.S. Pat. Nos. 5,871,921; 6,235,472; 5,866,337; and Japanese patent JP 4-262799; rolling circle probes being disclosed in Aono et al, JP4-262799; Lizardi, U.S. Pat. Nos. 5,854,033; 6,183,960; 6,344,239; molecular inversion probes being disclosed in Hardenbol et al (cited above) and in Willis et al, U.S. patent publication 2004/0101835; and linear amplification molecules being disclosed in Faham et al, U.S. patent publication 2003/0104459; all of which are incorporated herein by reference. Such probes are desirable because non-circularized probes can be digested with single stranded exonucleases thereby greatly reducing background noise due to spurious amplifications, and the like. In the case of molecular inversion probes (MIPs), padlock probes, and rolling circle probes, constructs for generating labeled target sequences are formed by circularizing a linear version of the probe in a template-driven reaction on a target polynucleotide followed by digestion of non-circularized polynucleotides in the reaction mixture, such as target polynucleotides, unligated probe, probe concatatemers, and the like, with an exonuclease, such as exonuclease I.

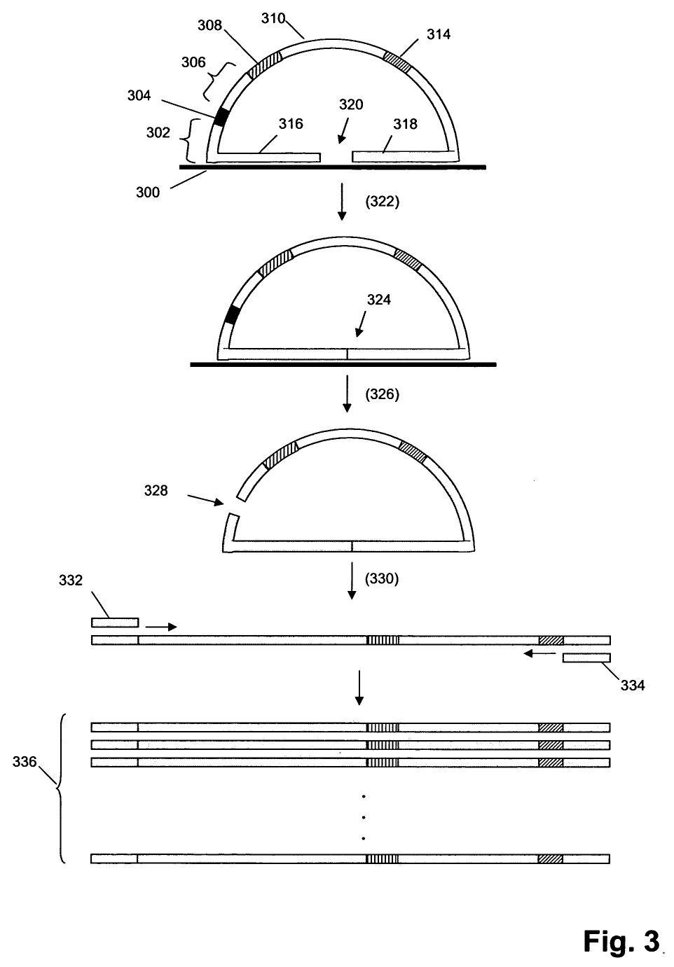

FIG. 3 illustrates a molecular inversion probe and how it can be used to generate an amplicon after interacting with a target polynucleotide in a sample. A linear version of the probe is combined with a sample containing target polynucleotide (300) under conditions that permit target-specific region 1 (316) and target-specific region 2 (318) to form stable duplexes with complementary regions of target polynucleotide (300). The ends of the target-specific regions may abut one another (being separated by a “nick”) or there may be a gap (320) of several (e.g. 1-10 nucleotides) between them. In either case, after hybridization of the target-specific regions, the ends of the two target specific regions are covalently linked by way of a ligation reaction or an extension reaction followed by a ligation reaction, i.e. a so-called “gap-filling” reaction. The latter reaction is carried out by extending with a DNA polymerase a free 3′ end of one of the target-specific regions so that the extended end abuts the end of the other target-specific region, which has a 5′ phosphate, or like group, to permit ligation. In one aspect, a molecular inversion probe has a structure as illustrated in FIG. 3. Besides target-specific regions (316 and 318), in sequence such a probe may include first primer binding site (302), cleavage site (304), second primer binding site (306), first tag-adjacent sequences (308) (usually restriction endonuclease sites and/or primer binding sites) for tailoring one end of a labeled target sequence containing oligonucleotide tag (310), and second tag-adjacent sequences (314) for tailoring the other end of a labeled target sequence. Alternatively, cleavage-site (304) may be added at a later step by amplification using a primer containing such a cleavage site. In operation, after specific hybridization of the target-specific regions and their ligation (322), the reaction mixture is treated with a single stranded exonuclease that preferentially digests all single stranded nucleic acids, except circularized probes. After such treatment, circularized probes are treated (326) with a cleaving agent that cleaves the probe between primer (302) and primer (306) so that the structure is linearized (330). Cleavage site (304) and its corresponding cleaving agent is a design choice for one of ordinary skill in the art. In one aspect, cleavage site (304) is a segment containing a sequence of uracil-containing nucleotides and the cleavage agent is treatment with uracil-DNA glycosylase followed by heating. After the circularized probes are opened, the linear product is amplified, e.g. by PCR using primers (332) and (334), to form amplicons (336). A multiplexed readout may be obtained from amplicon (336) by labeling and excising oligonucleotide tag (310) and specifically hybridizing the labeled tags to a microarray of tag complements, e.g. a GenFlex array (Affymetrix, Santa Clara, Calif.); a bead array (Illumina, San Diego, Calif.); or a fluid array, e.g. Chandler et al, U.S. Pat. No. 5,981,180 (Lumenix, Austin, Tex.).

Labeling Oligonucleotide Tags

Hybridization of oligonucleotide tags generated in accordance with the invention can be labeled in a variety of ways, including the direct or indirect attachment of fluorescent moieties, colorimetric moieties, chemiluminescent moieties, and the like. Many comprehensive reviews of methodologies for labeling DNA provide guidance applicable to generating labeled oligonucleotide tags of the present invention. Such reviews include Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); Wetmur, Critical Reviews in Biochemistry and Molecular Biology, 26: 227-259 (1991); and the like. Particular methodologies applicable to the invention are disclosed in the following sample of references: Fung et al, U.S. Pat. No. 4,757,141; Hobbs, Jr., et al U.S. Pat. No. 5,151,507; Cruickshank, U.S. Pat. No. 5,091,519. In one aspect, one or more fluorescent dyes are used as labels for the oligonucleotide tags, e.g. as disclosed by Menchen et al, U.S. Pat. No. 5,188,934 (4,7-dichlorofluorscein dyes); Begot et al, U.S. Pat. No. 5,366,860 (spectrally resolvable rhodamine dyes); Lee et al, U.S. Pat. No. 5,847,162 (4,7-dichlororhodamine dyes); Khanna et al, U.S. Pat. No. 4,318,846 (ether-substituted fluorescein dyes); Lee et al, U.S. Pat. No. 5,800,996 (energy transfer dyes); Lee et al, U.S. Pat. No. 5,066,580 (xanthene dyes): Mathies et al, U.S. Pat. No. 5,688,648 (energy transfer dyes); and the like. As used herein, the term “fluorescent signal generating moiety” means a signaling means which conveys information through the fluorescent absorption and/or emission properties of one or more molecules. Such fluorescent properties include fluorescence intensity, fluorescence life time, emission spectrum characteristics, energy transfer, and the like.

In particular, many schemes for generating copies of labeled oligonucleotide tags for hybridization to microarrays are disclosed in Namsaraev et al, International patent publication WO 2005/029040, which is incorporated herein by reference.

### Hybridization of Labeled Tag Sequences to Solid Phase Supports

Methods for hybridizing labeled oligonucleotide tags to microarrays, and like platforms, suitable for the present invention are well known in the art. Guidance for selecting conditions and materials for applying labeled target sequences to solid phase supports, such as microarrays, may be found in the literature, e.g. Wetmur, Crit. Rev. Biochem. Mol. Biol., 26: 227-259 (1991); DeRisi et al, Science, 278: 680-686 (1997); Chee et al, Science, 274: 610-614 (1996); Duggan et al, Nature Genetics, 21: 10-14 (1999); Schena, Editor, Microarrays: A Practical Approach (IRL Press, Washington, 2000); Freeman et al, Biotechniques, 29: 1042-1055 (2000); and like references. Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference. Hybridization conditions typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will stably hybridize to a perfectly complementary target sequence, but will not stably hybridize to sequences that have one or more mismatches. The stringency of hybridization conditions depends on several factors, such as probe sequence, probe length, temperature, salt concentration, concentration of organic solvents, such as formamide, and the like. How such factors are selected is usually a matter of design choice to one of ordinary skill in the art for any particular embodiment. Usually, stringent conditions are selected to be about 5° C. lower than the Tₘ for the specific sequence for particular ionic strength and pH. Exemplary hybridization conditions include salt concentration of at least 0.01 M to no more than 1 M Na ion concentration (or other salts) at a pH 7.0 to 8.3 and a temperature of at least 25° C. Additional exemplary hybridization conditions include the following: 5×SSPE (750 mM NaCl, 50 mM sodium phosphate, 5 mM EDTA, pH 7.4).

Exemplary hybridization procedures for applying labeled oligonucleotide tags to a GenFlex™ microarray (Affymetrix, Santa Clara, Calif.) is as follows: denatured labeled target sequence at 95-100° C. for 10 minutes and snap cool on ice for 2-5 minutes. The microarray is pre-hybridized with 6×SSPE-T (0.9 M NaCl 60 mM NaH₂,PO₄, 6 mM EDTA (pH 7.4), 0.005% Triton X-100)+0.5 mg/ml of BSA for a few minutes, then hybridized with 120 μL hybridization solution (as described below) at 42° C. for 2 hours on a rotisserie, at 40 RPM. Hybridization Solution consists of 3M TMACL (Tetranethylammonium. Chloride), 50 mM MES ((2-[N-Morpholino]ethanesulfonic acid) Sodium Salt) (pH 6.7), 0.01% of Triton X-100, 0.1 mg/ml of Herring Sperm DNA, optionally 50 pM of fluorescein-labeled control oligonucleotide, 0.5 mg/ml of BSA (Sigma) and labeled target sequences in a total reaction volume of about 120 μL. The microarray is rinsed twice with 1×SSPE-T for about 10 seconds at room temperature, then washed with 1×SSPE-T for 15-20 minutes at 40° C. on a rotisserie, at 40 RPM. The microarray is then washed 10 times with 6×SSPE-T at 22° C. on a fluidic station (e.g. model FS400, Affymetrix, Santa Clara, Calif.). Further processing steps may be required depending on the nature of the label(s) employed, e.g. direct or indirect. Microarrays containing labeled target sequences may be scanned on a confocal scanner (such as available commercially from Affymetrix) with a resolution of 60-70 pixels per feature and filters and other settings as appropriate for the labels employed. GeneChip Software (Affymetrix) may be used to convert the image files into digitized files for further data analysis.

### Detection of Hybridized Labeled Tag Sequences

Labeled oligonucleotide tags of the invention are detected by specifically hybridizing them to one or more solid supports containing end-attached tag complements, usually in the form of a microarray of spatially discrete hybridization sites. Instruments for measuring optical signals, especially fluorescent signals, from labeled tags hybridized to targets on a microarray are described in the following references which are incorporated by reference: Stem et al, PCT publication WO 95/22058; Resnick et al, U.S. Pat. No. 4,125,828; Karnaukhov et al, U.S. patent ,354,114; Trulson et al, U.S. Pat. No. 5,578,832; Pallas et al, PCT publication WO 98/53300; and the like.

The above teachings are intended to illustrate the invention and do not by their details limit the scope of the claims of the invention. While preferred illustrative embodiments of the present invention are described, it will be apparent to one skilled in the art that various changes and modifications may be made therein without departing from the invention, and it is intended in the appended claims to cover all such changes and modifications that fall within the true spirit and scope of the invention.

1. A method of determining relative amounts of target polynucleotides in a population, the method comprising the steps of:
providing for each target polynucleotide a plurality of probes, each probe of the same plurality being specific for the same target polynucleotide and each probe of the same or different plurality having a different oligonucleotide tag, the oligonucleotide tags of all the pluralities belonging to the same minimally cross-hybridizing set;
combining in a reaction mixture the pluralities of probes with the population so that substantially every target polynucleotide specifically hybridizes to one or more probes of its corresponding plurality and so that probes specifically hybridized to a target polynucleotide are enzymatically modified to form selectable probes;
removing a sample of selectable probes from the reaction mixture;
amplifying and labeling the oligonucleotide tags of the sample;
specifically hybridizing the labeled oligonucleotide tags to their respective tag complements on one or more solid phase supports having addressable hybridization sites; and
determining for each plurality of probes a proportion of hybridization sites on the one or more solid phase supports that contain labeled oligonucleotide probes to give a relative amount of the corresponding target polynucleotide in the population.

2. The method of claim 1 wherein each of said target polynucleotides is a genetic locus or a gene expression product.

3. The method of claim 2 wherein said one or more solid phase supports are a set of microbeads such that each microbead in the set has a distinct address.

4. The method of claim 3 wherein each of said probes is a molecular inversion probe and wherein each of said selectable probes is a circularized molecular inversion probe.

5. The method of claim 2 wherein said one or more solid phase supports is a microarray.

6. The method of claim 5 wherein each of said probes is a molecular inversion probe and wherein each of said selectable probes is a circularized molecular inversion probe.

7. The method of claim 6 wherein each of said pluralities of said probes is in the range of from 10 to 1000.

8. A probe composition for detecting one or more target polynucleotides in a sample, the composition comprising a plurality of probes for each target polynucleotide, each probe of the same plurality being specific for the same target polynucleotide and each probe of the same or different plurality having a different oligonucleotide tag, wherein each probe specifically hybridizes to a region of a target polynucleotide and the oligonucleotide tags each belong to the same minimally cross-hybridizing set.

9. The probe composition of claim 8 wherein each of said pluralities of probes is in the range of from 10 to 1000.

10. The probe composition of claim 9 wherein said one or more target polynucleotides are from two to five hundred target polynucleotides, with the proviso that said probes of all of said pluralities number less than 50,000.

11. A method of determining absolute concentrations of target polynucleotides in a test sample, the method comprising the steps of:
providing one or more nucleic acid standards each having a concentration;
providing for each target polynucleotide and each nucleic acid standard a plurality of probes, each probe of the same plurality being specific for the same target polynucleotide or the same nucleic acid standard and each probe of the same or different plurality having a different oligonucleotide tag, the oligonucleotide tags of all the pluralities belonging to the same minimally cross-hybridizing set;
combining in a reaction mixture the pluralities of probes with the test sample so that substantially every target polynucleotide and nucleic acid standard specifically hybridizes to one or more probes of its corresponding plurality and so that probes specifically hybridized to a target polynucleotide or a nucleic acid standard are enzymatically modified to form selectable probes;
removing a sample of selectable probes from the reaction mixture;
amplifying and labeling the oligonucleotide tags of the sample;
specifically hybridizing the labeled oligonucleotide tags to their respective tag complements on one or more solid phase supports having addressable hybridization sites; and
determining an absolute concentration of each target polynucleotide in the test sample by comparing a number of hybridization sites generating signals from a specifically hybridized oligonucleotide tag of a target polynucleotide to a number of hybridization sites generating signals from one or more nucleic acid standards and their respective concentrations.

12. The method of claim 1 wherein each of said target polynucleotides is a genetic locus or a gene expression product.

13. The method of claim 2 wherein said one or more solid phase supports are a set of microbeads such that each microbead in the set has a distinct address.

14. The method of claim 3 wherein each of said probes is a molecular inversion probe and wherein each of said selectable probes is a circularized molecular inversion probe.

15. The method of claim 2 wherein said one or more solid phase supports is a microarray.

16. The method of claim 5 wherein each of said probes is a molecular inversion probe and wherein each of said selectable probes is a circularized molecular inversion probe.

17. The method of claim 6 wherein each of said pluralities of said probes is in the range of from 10 to 1000.
